(19) United States
(12) Patent Application Publication
(10) Pub. No.: US 2017/0053081 A1
(43) Pub. Date: Feb. 23, 2017

(54) METHODS AND MATERIALS FOR CHARACTERIZING INTESTINAL BARRIER FUNCTION

(71) Applicant: THE ARIZONA BOARD OF REGENTS ON BEHALF OF THE UNIVERSITY OF ARIZONA, Tucson, AZ (US)

(72) Inventors: Katri TYPPO, Tucson, AZ (US); Fayez K. GHISHAN, Tucson, AZ (US); Pawel R. KIELA, Tucson, AZ (US)

(21) Appl. No.: 15/308,286

(22) PCT Filed: Apr. 30, 2015
(86) PCT No.: PCT/US2015/028515

§ 371 Date: Nov. 1, 2016

Related U.S. Application Data
(60) Provisional application No. 61/987,952, filed on May 2, 2014.

(51) Int. Cl. G06F 19/00; G01N 33/68; A61K 9/00; A23L 33/00; A61K 38/01

(52) U.S. Cl. CPC G06F 19/345; A23L 33/40; A61K 38/018; A61K 9/0053; A61K 38/01; G01N 33/6893; A23V 2002/00; G01N 2800/06

(57) ABSTRACT

The present invention the present invention provides methods and materials related to the characterization of intestinal barrier function, and related methods for preventing and/or treating intestinal barrier dysfunction. In particular, the present invention provides methods and materials for characterizing intestinal barrier function within mammals (e.g., humans) through determining the level, presence, and/or frequency of biomarkers for intestinal function (e.g., functional enterocyte mass, enterocyte integrity, paracellular tight junction function, gut inflammation) within a biological sample.

The present application claims priority to U.S. Provisional Patent Application Ser. No. 61/987,952, filed May 2, 2014, the disclosure of which is herein incorporated by reference in its entirety.

### STATEMENT REGARDING FEDERALLY SPONSORED RESEARCH OR DEVELOPMENT

This invention was made with government support under K12 HD047349 awarded by the National Institutes of Health. The government has certain rights in the invention.

### FIELD OF THE INVENTION

The present invention the present invention provides methods and materials related to the characterization of intestinal barrier function, and related methods for preventing and/or treating intestinal barrier dysfunction. In particular, the present invention provides methods and materials for characterizing intestinal barrier function within mammals (e.g., humans) through determining the level, presence, and/or frequency of biomarkers for intestinal function (e.g., functional enterocyte mass, enterocyte integrity, paracellular tight junction function, gut inflammation) within a biological sample.

### BACKGROUND OF THE INVENTION

Intestinal barrier dysfunction can result in translocation of bacterial products into the bloodstream, decreased gastrointestinal intraepithelial lymphocyte populations, and loss of IgA mediated mucosal immunity in the gastrointestinal and respiratory tract, negatively affecting host immune response while also increasing exposure to pathogens (see, e.g., Sacks, et al., AmJPathol 2010; 176:2283-91; Nose, et al., JInterferon Cytokine Res 2010; 30:67-80; Johnson, et al., AnnSurg 2003; 237:565-73). One single-center study of 61 children with CHD correlated FABP2 levels with post-operative endotoxemia and organ dysfunction, supporting the role of bacterial translocation and immune dysfunction in the pathogenesis of post-operative sepsis (see, e.g., Pathan, et al., American journal of respiratory and critical care medicine 2011; 184:1261-9). Maintenance of intestinal barrier function may have downstream effects on both host burden of exposure to pathogens, and immune function. Restoration of intestinal epithelial barrier function is associated with restored local and remote organ mucosal immunity in animal models of critical illness and offers a potential target to reduce post-operative hospital-acquired infection risk and organ dysfunction (see, e.g., Kudsk, et al., JParenterEnteral Nutr 2000; 24:270-4). Several potential therapies, such as human recombinant lactoferrin, exist to maintain intestinal epithelial barrier function but require further study as a therapy to prevent post-operative intestinal barrier dysfunction (see, e.g., Guntupalli, et al., Critical care medicine 2013; 41:706-16).

Improved methods for characterizing intestinal barrier function and intestinal barrier dysfunction are needed. In addition, improved methods for treating and preventing intestinal barrier dysfunction are needed.

### SUMMARY

Intake of higher percent of energy via the enteral route is associated with improved 60-day survival during pediatric critical illness. And yet, despite guideline recommended early enteral nutrition (EN) protocols, median daily calorie delivery remains 40-75% of goal. Children fail to receive goal calories due to feeding intolerance, interruptions in EN, and due to physician concerns for safety of EN in patients with hemodynamic or respiratory instability. No objective diagnostic tools exist to inform physician decisions with regard to initiation, advancement, or withdrawal of EN for children with hemodynamic instability and respiratory failure.

Previous research with children after cardiac surgery demonstrated that serum biomarkers of intestinal barrier function were found to be improved with delivery of small dose EN, and to correlate with symptoms of feeding intolerance. Infants diagnosed with necrotizing enterocolitis, who have a rise in these same biomarker levels after re-introduction of EN were found to have worse outcomes. In these series of patients, biomarkers of intestinal barrier function improve with early EN, correlate with symptoms of feeding intolerance, and are predictive of gastrointestinal complications. In such experiments, intestinal barrier function was also modulated by dose and type of vasoactive infusion, and by mean airway pressure. These data indicate that non-invasive assessment of intestinal barrier function guides safe and optimized EN delivery in children with hemodynamic instability and respiratory failure.

The absence of EN results in impaired intestinal barrier function in humans and animals, an effect worsened by Parenteral Nutrition (PN) administration. Intestinal barrier dysfunction is linked to sepsis, hospital-acquired infection risk, and to immunoregulatory responses triggering organ dysfunction. Mice with ischemia reperfusion injury, given PN with 15% of goal EN, retained intestinal barrier function. Determining minimum dose of EN to preserve the intestinal barrier could reduce hospital-acquired infection risk and morbidity after pediatric critical illness for patients who fail to tolerate full EN.

Accordingly, the present invention the present invention provides methods and materials related to the characterization of intestinal barrier function, and related methods for preventing and/or treating intestinal barrier dysfunction. In particular, the present invention provides methods and materials for characterizing intestinal barrier function within mammals (e.g., humans) through determining the level, presence, and/or frequency of biomarkers for intestinal function (e.g., functional enterocyte mass, enterocyte integrity, paracellular tight junction function, gut inflammation) within a biological sample.

In certain embodiments, the present invention provides methods for characterizing intestinal barrier function in a subject comprising a) providing reagents necessary for determining the level, presence, and/or frequency of two or more biomarkers for intestinal barrier function, and an algorithm configured to receive information regarding the level, presence and/or frequency of two or more biomarkers for intestinal barrier function within a biological sample obtained from a subject, and configured to compare received information regarding the level, presence and/or frequency of two or more biomarkers for intestinal barrier function with established norms for intestinal barrier function, and based upon such comparison, characterize the intestinal barrier function for the subject; b) obtaining a biological sample from a subject; c) determining the level, presence, and/or frequency of two or more biomarkers indicative of intestinal barrier function within the biological sample; d) inputting the determined level, presence, and/or frequency of the two or more biomarkers into the algorithm; and e) characterizing the intestinal barrier function of the subject with the algorithm. In some embodiments, steps b), c), d) and e) are repeated for purposes of monitoring the intestinal barrier function for a subject.

Such methods are not limited to a particular type of subject. In some embodiments, the subject is a human subject. In some embodiments, the subject is a human critical care patient that is being closely monitored, undergoing surgical repair or palliation of congenital heart disease (CHD), and/or undergoing cardiopulmonary bypass surgery.

Such methods are not limited to particular biomarkers for intestinal barrier function. In some embodiments, the two or more biomarkers for intestinal barrier function are selected from the group consisting of a biomarker for functional enterocyte mass, a biomarker for enterocyte integrity, a biomarker for paracellular tight junction function, and a biomarker for gut inflammation. In some embodiments, the biomarker for functional enterocyte mass is citrulline. In some embodiments, the biomarker for enterocyte integrity is a fatty-acid binding protein (FABP) (e.g., FABP2 (also known as I-FABP)). In some embodiments, the biomarker for paracellular tight junction function is claudin-3. In some embodiments, the biomarker for gut inflammation is calprotectin. In some embodiments, the methods further comprise conducting dual sugar permeability testing (DSPT) for purposes of assessing intestinal barrier function.

Such methods are not limited to a particular type of biological sample. In some embodiments, the biological sample is selected from the group consisting of a blood sample, a plasma sample, a serum sample, a fecal sample, and a urine sample.

Such methods are not limited to particular established norms for intestinal barrier function. In some embodiments, the established norm for intestinal barrier function is one or more established norm selected from the group consisting of an established norm for normal intestinal barrier function specific for the received information regarding the level, presence and/or frequency of two or more biomarkers, an established norm for compromised intestinal barrier function for the received information regarding the level, presence and/or frequency of two or more biomarkers, and an established norm for neither healthy nor compromised intestinal barrier function for the received information regarding the level, presence and/or frequency of two or more biomarkers. In some embodiments, the established norm is specific for a human critical care patient that is being closely monitored. In some embodiments, the established norm is specific for a medical procedure selected from the group consisting of surgical repair or palliation of congenital heart disease (CHD) and cardiopulmonary bypass surgery.

In some embodiments, the determining the level, presence, and/or frequency of two or more biomarkers indicative of intestinal barrier function within the biological sample is accomplished with an assay configured to determine the level, presence, and/or frequency of the two or more biomarkers indicative of intestinal barrier function within the biological sample. In some such embodiments, the biological sample is urine and the assay is a dip-stick assay. In some embodiments, the assay is configured to visually indicate the subject's intestinal barrier function characterization. In some embodiments, the assay is configured to visually indicate whether or not enteral and/or parenteral nutrition should be administered to the subject.

In certain embodiments, the present invention provides methods for treating intestinal barrier dysfunction in a subject, comprising characterizing a subject's intestinal barrier function with the described methods for characterizing intestinal barrier function in a subject, and administering enteral and/or parenteral nutrition to the subject if the subject's intestinal barrier function is characterized as non-healthy. In some embodiments, parenteral nutrition in initiated at the same time as enteral nutrition. In some embodiments, the concentration of parenteral nutrition is approximately 75% of total calories and the concentration of enteral nutrition is approximately 25% of total calories at the beginning of the treatment. In some embodiments, the concentration of parenteral nutrition is decreased over time and the concentration of enteral nutrition is increased over time such that at the end of approximately 1 week, all of the nutrition is enteral. In some embodiments, the subject is diagnosed with acute respiratory distress or failure.

In certain embodiments, the present invention provides methods of preventing intestinal barrier dysfunction in a subject, comprising monitoring a subject's intestinal barrier function with the described methods for characterizing intestinal barrier function in a subject, and administering enteral nutrition to the subject if the subject's intestinal barrier function is trending from healthy to inbetween healthy and non-healthy.

In certain embodiments, the present invention provides kits for characterizing intestinal barrier function in a subject comprising reagents necessary for determining the level, presence, and/or frequency of two or more biomarkers for intestinal barrier function, and an algorithm configured to receive information regarding the level, presence and/or frequency of two or more biomarkers for intestinal barrier function within a biological sample obtained from a subject, and configured to compare received information regarding the level, presence and/or frequency of two or more biomarkers for intestinal barrier function with established norms for intestinal barrier function, and based upon such comparison, characterize the intestinal barrier function for the subject.

In certain embodiments, the present invention provides kits for characterizing intestinal barrier function in a subject comprising an assay having thereon reagents necessary for determining the level, presence, and/or frequency of two or more biomarkers for intestinal barrier function, and an algorithm configured to receive information regarding the level, presence and/or frequency of two or more biomarkers for intestinal barrier function within a biological sample obtained from a subject, and configured to compare received information regarding the level, presence and/or frequency of two or more biomarkers for intestinal barrier function with established norms for intestinal barrier function, and based upon such comparison, characterize the intestinal barrier function for the subject. In some embodiments, the biological sample is urine. In some embodiments, the assay is a dip-stick assay. In some embodiments, assay is configured to visually indicate the subject's intestinal barrier dysfunction characterization. In some embodiments, the assay is configured to visually indicate whether or not enteral and/or parenteral nutrition should be administered to the subject.

In certain embodiments, the present invention provides methods for generating a subject's risk profile for developing intestinal barrier dysfunction, comprising a) providing reagents necessary for determining the level, presence, and/or frequency of two or more biomarkers for intestinal barrier function, and an algorithm configured to receive information regarding the level, presence and/or frequency of two or more biomarkers for intestinal barrier function within a biological sample obtained from a subject, and configured to compare received information regarding the level, presence and/or frequency of two or more biomarkers for intestinal barrier function with established norms for intestinal barrier dysfunction, and based upon such comparison, generating a risk profile for developing intestinal barrier dysfunction for the subject; b) obtaining a biological sample from a subject; c) determining the level, presence, and/or frequency of two or more biomarkers indicative of intestinal barrier function within the biological sample; d) inputting the determined level, presence, and/or frequency of the two or more biomarkers into the algorithm; and e) generating a risk profile for developing intestinal barrier dysfunction for the subject with the algorithm. In some embodiments, steps b), c), d) and e) are repeated for purposes of monitoring the subject's risk profile for developing intestinal barrier dysfunction.

Such methods are not limited to a particular type of subject. In some embodiments, the subject is a human critical care patient that is being closely monitored, undergoing surgical repair or palliation of congenital heart disease (CHD), and/or undergoing cardiopulmonary bypass surgery

Such methods are not limited to particular biomarkers for intestinal barrier function. In some embodiments, the two or more biomarkers for intestinal barrier function are selected from the group consisting of a biomarker for functional enterocyte mass, a biomarker for enterocyte integrity, a biomarker for paracellular tight junction function, and a biomarker for gut inflammation. In some embodiments, the biomarker for functional enterocyte mass is citrulline. In some embodiments, the biomarker for enterocyte integrity is a fatty-acid binding protein (FABP) (e.g., FABP2). In some embodiments, the biomarker for paracellular tight junction function is claudin-3. In some embodiments, the biomarker for gut inflammation is calprotectin.

Such methods are not limited to a particular type of biological sample. In some embodiments, the biological sample is selected from the group consisting of a blood sample, a plasma sample, a serum sample, a fecal sample, and a urine sample.

Such methods are not limited to a particular type of established norm for intestinal barrier dysfunction. In some embodiments, the established norm for intestinal barrier dysfunction is one or more established norm selected from the group consisting of an established norm for very low risk for developing intestinal barrier dysfunction specific for the received information regarding the level, presence and/or frequency of two or more biomarkers, an established norm for low risk for developing intestinal barrier dysfunction specific for the received information regarding the level, presence and/or frequency of two or more biomarkers, an established norm for moderate risk for developing intestinal barrier dysfunction specific for the received information regarding the level, presence and/or frequency of two or more biomarkers, an established norm for high risk for developing intestinal barrier dysfunction specific for the received information regarding the level, presence and/or frequency of two or more biomarkers, and an established norm for very high risk for developing intestinal barrier dysfunction specific for the received information regarding the level, presence and/or frequency of two or more biomarkers. In some embodiments, the established norm is specific for a human critical care patient that is being closely monitored. In some embodiments, the established norm is specific for medical procedure selected from the group consisting of surgical repair or palliation of congenital heart disease (CHD) and cardiopulmonary bypass surgery.

In some embodiments, the determining the level, presence, and/or frequency of two or more biomarkers indicative of intestinal barrier function within the biological sample is accomplished with an assay configured to determine the level, presence, and/or frequency of the two or more biomarkers indicative of intestinal barrier function within the biological sample. In some such embodiments, the biological sample is urine and the assay is a dip-stick assay. In some embodiments, the assay is configured to visually indicate the subject's risk profile for developing intestinal barrier dysfunction. In some embodiments, the assay is configured to visually indicate whether or not enteral and/or parenteral nutrition should be administered to the subject.

In certain embodiments, the present invention provides methods for treating intestinal barrier dysfunction in a subject, comprising characterizing a subject's intestinal barrier function with the described methods for generating a subject's risk profile for developing intestinal barrier dysfunction and administering enteral nutrition to the subject if the subject's intestinal barrier function is characterized as non-healthy.

In certain embodiments, the present invention provides methods for preventing intestinal barrier dysfunction in a subject, comprising monitoring a subject's intestinal barrier function with the described methods for generating a subject's risk profile for developing intestinal barrier dysfunction, and administering enteral and/or parenteral nutrition to the subject if the subject's intestinal barrier function is trending from healthy to inbetween healthy and non-healthy.

In certain embodiments, the present invention provides kits for generating a subject's risk profile for developing intestinal barrier dysfunction, comprising reagents necessary for determining the level, presence, and/or frequency of two or more biomarkers for intestinal barrier function, and an algorithm configured to receive information regarding the level, presence and/or frequency of two or more biomarkers for intestinal barrier function within a biological sample obtained from a subject, and configured to compare received information regarding the level, presence and/or frequency of two or more biomarkers for intestinal barrier function with established norms for intestinal barrier dysfunction, and based upon such comparison, generating a risk profile for developing intestinal barrier dysfunction for the subject.

In certain embodiments, the present invention provides kits for generating a subject's risk profile for developing intestinal barrier dysfunction, comprising an assay having thereon reagents necessary for determining the level, presence, and/or frequency of two or more biomarkers for intestinal barrier function, and an algorithm configured to receive information regarding the level, presence and/or frequency of two or more biomarkers for intestinal barrier function within a biological sample obtained from a subject, and configured to compare received information regarding the level, presence and/or frequency of two or more biomarkers for intestinal barrier function with established norms for intestinal barrier dysfunction, and based upon such comparison, generating a risk profile for developing intestinal barrier dysfunction for the subject. In some embodiments, the biological sample is a blood sample or a urine sample. In some embodiments, the assay is a dip-stick assay. In some embodiments, the assay is configured to visually indicate the subject's risk profile for developing intestinal barrier dysfunction. In some embodiments, the assay is configured to visually indicate whether or not enteral and/or parenteral nutrition should be administered to the subject.

Additional embodiments will be apparent to persons skilled in the relevant art based on the teachings contained herein.

### BRIEF DESCRIPTION OF THE DRAWINGS

FIG. 1 shows change in non-invasive intestinal barrier function biomarkers and regulatory cytokines after cardiopulmonary bypass. FABP2, Intestinal Fatty Acid Binding Protein; IL-10, Interleukin 10; IL-4, Interleukin 4; IL-6, Interleukin 6; TNF-α, Tumor Necrosis Factor α; INF-γ, Interferon γ. Repeated claudin 3 and Lactulose/Mannitol ratios were significantly associated (p<0.01) and rise remote from cardiopulmonary bypass (CPB).

FIG. 2 shows plasma FABP2 and citrulline change with vasoactive-inotrope score. FABP2, Intestinal Fatty Acid Binding Protein; VIS, vasoactive infusion score.

FIG. 3 shows a model of intestinal barrier function.

FIG. 4 shows an exemplary model of the effect of PN on intestinal barrier function. A) Model where PN causes lack of microvillus height and loss of tight epithelial junction function. B) Model where early PN, in combination with early EN, enhances epithelial barrier function.

FIG. 5 shows that the use of early PN to achieve goal energy and protein as a supplement to EN causes an early (within 48 hours) and significant decrease in plasma FABP2 concentrations which persists throughout the first week of PICU hospitalization.

### DETAILED DESCRIPTION OF THE INVENTION

Children undergoing surgical repair or palliation of congenital heart disease (CHD) are at high risk of post-operative organ dysfunction, sepsis, and gastrointestinal complications (see, e.g., Pathan, et al., American journal of respiratory and critical care medicine 2011; 184:1261-9; Wheeler, et al., World JPediatrCongenitHeart Surg 2011; 2:393-9). Intestinal ischemia-reperfusion injury occurring after cardiopulmonary bypass (CPB) worsens intestinal epithelial barrier function, permitting bacteria or bacterial products to translocate across the intestinal barrier and into the bloodstream (see, e.g., Pathan, et al., American journal of respiratory and critical care medicine 2011; 184:1261-9). Post-operative intestinal epithelial barrier dysfunction is associated with the development of organ dysfunction, sepsis, and gastrointestinal complications (see, e.g., Pathan, et al., American journal of respiratory and critical care medicine 2011; 184:1261-9; Holmes, et al., Journal of surgical research 2001; 100:192-6). In murine models, intestinal barrier dysfunction is also associated with loss of local and remote organ immune function; specifically with loss of gastrointestinal and respiratory secretory IgA, an effect mediated by a reduced number and complexity of intestinal intraepithelial lymphocyte populations (see, e.g., Wildhaber, et al., JSurgRes 2005; 123:8-16; Sacks, et al., NutrClinPract 2003; 18:483-8). Intestinal epithelial barrier dysfunction after CPB has downstream effects on nutrient absorption, exposure to pathogens and their products, and immune regulation.

Non-invasive assessment of intestinal epithelial barrier function or intestinal barrier function is possible using plasma markers, which reflect downstream structural and functional changes to the intestinal barrier, and through functional sugar permeability testing (see, e.g., Grootjans, et al., AmJPathol 2010; 176:2283-91; Grootjans, et al., World JGastrointestSurg 2010; 2:61-9). Intestinal Fatty Acid Binding Protein (FABP2) is a cytosolic protein found primarily in mature enterocytes in the small intestine has been used as a biomarker of early intestinal ischemia and injury in children and adults after cardiopulmonary bypass (see, e.g., Pathan, et al., American journal of respiratory and critical care medicine 2011; 184:1261-9; Holmes, et al., Journal of surgical research 2001; 100:192-6). FABP2 plasma concentration correlates with plasma endotoxin levels and histological phase of intestinal epithelial injury (see e.g., Grootjans, et al., AmJPathol 2010; 176:2283-91; Thuijls, et al., AnnSurg 2011; 253:303-8). Functional enterocyte mass in turn, can be measured by the levels of circulating citrulline, a non-essential amino acid in humans (see, e.g., Bailly-Botuha, et al., Pediatric research 2009; 65:559-63). Plasma levels of citrulline reflect enterocyte citrulline synthesis, and correlate with functional enterocyte mass in stem cell transplant patients, and in pediatric short bowel syndrome (see, e.g., Rhoads, et al., Journal of pediatrics 2005; 146:542-7; Merlin, et al., Pediatric transplantation 2013; 17:683-7). Transmembrane tight junction proteins such as claudins are the primary determinants of gastrointestinal paracellular barrier integrity (see, e.g., Patel, et al., AmJPathol 2012; 180:626-35; Turner, et al., NatRevImmunol 2009; 9:799-809). Plasma claudin 3 is a non-invasive marker for early intestinal tight junction loss, and is localized to the epithelial tight junctions (see, e.g., Patel, et al., AmJPathol 2012; 180:626-35). Some recent studies have shown a strong relationship between intestinal tight junction loss and claudin 3 plasma levels in a rat hemorrhagic shock model, during developmental maturation of the gastrointestinal tract, and in children undergoing surgery (see, e.g., Patel, et al., AmJPathol 2012; 180:626-35; Thuijls, et al., Journal of clinical gastroenterology 2010; 44:e14-9; Thuijls, et al., Annals of surgery 2010; 251:1174-80). Dual sugar permeability testing (DSPT) relies on the differential intestinal paracellular and cellular permeability of larger (lactulose) and smaller (mannitol) molecules (see, e.g., Rao, et al., AmJPhysiol GastrointestLiver Physiol 2011; 301:G919-G28). Simultaneous co-ingestion of lactulose and mannitol are used as controls for gastric emptying, intestinal fluid volume, gastrointestinal transit time, and renal excretion which are thought to affect each molecule equally (see, e.g., Rao, et al., AmJPhysiol GastrointestLiver Physiol 2011; 301:G919-G28). The ratio of urinary excretion reflects small intestinal permeability.

Previously thought to be static, intestinal epithelial barrier function or intestinal barrier function is adaptable throughout the gastrointestinal tract and is regulated by diverse extracellular stimuli, including nutrients, medications, commensal and pathogenic organisms, and cytokines (see, e.g., Patel, et al., AmJPathol 2012; 180:626-35; Feng, et al., Annals of the New York Academy of Sciences 2012; 1258:71-7; Jin, et al., Gut 2010; 59:186-96; Ohta, et al., American journal of surgery 2003; 185:79-85; Song, et al., World journal of gastroenterology: WJG 2005; 11:3701-9). Intestinal barrier function after repair or palliation of CHD offers a novel target for the prevention of post-operative sepsis, and organ dysfunction; sources of ongoing morbidity in this patient population (see, e.g., Pathan, et al., American journal of respiratory and critical care medicine 2011; 184:1261-9; Wheeler, et al., World JPediatrCongenitHeart Surg 2011; 2:393-9).

Experiments conducted during the course of developing embodiments for the present invention aimed at understanding how post-operative fluid, nutritional, and hemodynamic management affected immediate and late post-operative intestinal barrier function. A panel of plasma biomarkers was used to reflect intestinal epithelial cellular and paracellular structure (FABP2 and claudin 3), as well as cellular and paracellular function (citrulline and DSPT). In particular, twenty children aged newborn to 18 years undergoing repair or palliation of congenital heart disease with CPB were examined. Plasma FABP2, claudin 3, and citrulline were measured after induction of general anesthesia, and at 6, 12, 24, 48, and 120 hours post-operatively. It was shown that children undergoing CPB for repair or palliation of congenital heart disease are at risk for intestinal injury and often present with altered intestinal barrier function pre-operatively. Vasopressin, commonly used to maintain mean arterial blood pressure post-operatively, was shown to increase the risk for intestinal barrier dysfunction, especially when used in combination with epinephrine.

Accordingly, the present invention the present invention provides methods and materials related to the characterization of intestinal barrier function, and related methods for preventing and/or treating intestinal barrier dysfunction. In particular, the present invention provides methods and materials for characterizing intestinal barrier function within mammals (e.g., humans) through determining the level, presence, and/or frequency of biomarkers for intestinal function (e.g., functional enterocyte mass, enterocyte integrity, paracellular tight junction function, gut inflammation) within a biological sample.

The methods and materials related to the characterization of intestinal barrier function, and related methods for preventing and/or treating intestinal barrier dysfunction are not limited to use with a particular type of subject. Indeed, the term “subject” includes animals, preferably mammals, including humans. In a preferred embodiment, the subject is a primate. In an even more preferred embodiment, the subject is a human. In some embodiments, the subject is a pediatric human while in other embodiments, the subject is an adult human. In some embodiments, the subject is at risk for intestinal barrier dysfunction. In some embodiments, the established norm is specific for a human critical care patient that is being closely monitored. In some embodiments, the subject is undergoing a challenge to intestinal barrier function (e.g., a subject undergoing surgical repair or palliation of congenital heart disease (CHD); a subject undergoing cardiopulmonary bypass surgery; a human critical care patient that is being closely monitored).

The present invention is not limited to particular methods for characterizing intestinal barrier function in a subject (e.g., a human subject). In some embodiments, such methods involve determining the level, presence, and/or frequency of biomarkers for intestinal barrier function in a biological sample obtained from a subject. Such methods are not limited to determining the level, presence, and/or frequency of specific biomarkers for intestinal barrier function. In some embodiments, the biomarkers are indicative of intestinal barrier function. Examples of biomarkers that are indicative of intestinal barrier function include, but are not limited to, biomarkers for functional enterocyte mass, biomarkers for enterocyte integrity, biomarkers for paracellular tight junction function, and biomarkers for gut inflammation. In some embodiments, the level, presence, and/or frequency of intestinal barrier function biomarkers provides functional information as to a subject's intestinal barrier function (e.g., its functional ability in relation to an established norm (e.g., a healthy intestinal barrier function, a compromised intestinal barrier function, etc).

Such methods are not limited to determining the level, presence, and/or frequency of a particular number of biomarkers for intestinal barrier function for purposes of characterizing a subject's intestinal barrier function. Indeed, while determining the level, presence, and/or frequency of one intestinal barrier function biomarker may be used to characterize a subject's intestinal barrier function, preferred embodiments of the present invention entail the use of a plurality (e.g., two or more, three or more, four or more, etc.) intestinal barrier function biomarkers. Indeed, the use of a plurality of intestinal barrier function biomarkers provides a more reliable characterization of a subject's intestinal barrier function.

In preferred embodiments, the level, presence, and/or frequency of biomarkers for functional enterocyte mass, biomarkers for enterocyte integrity, and biomarkers for paracellular tight junction function are determined within methods for characterizing a subject's intestinal barrier function. In some embodiments, the level, presence, and/or frequency of biomarkers for gut inflammation are further determined along with biomarkers for functional enterocyte mass, biomarkers for enterocyte integrity, and biomarkers for paracellular tight junction function within methods for characterizing a subject's intestinal barrier function. In some embodiments, such methods further comprise conducting dual sugar permeability testing (DSPT).

Such methods are not limited to specific biomarkers for functional enterocyte mass. In some embodiments, the level, presence, and/or frequency of citrulline within a subject's biological sample (e.g., plasma sample, serum sample, blood sample, urine sample, fecal sample) is used as a biomarker for functional enterocyte mass. Citrulline is an amino acid involved in intermediary metabolism and that is not incorporated in proteins. Circulating citrulline is mainly produced by enterocytes of the small bowel and, as such, is a biomarker of remnant small bowel mass and function (see, e.g., Crenn, et al., Clin. Nutr. 2008 27(3):328-39; Jianfeng, et al., J. Surg. Res. 2005 127(2):177-82; Karilik, et al., Bone Marrow Transplantation 49, 449-450 (March 2014)). The methods are not limited to a particular manner of determining the level, presence, and/or frequency of citrulline expression/concentration within a subject's biological sample (see, e.g., Examples I and II).

Such methods are not limited to specific biomarkers for enterocyte integrity. In some embodiments, the level, presence, and/or frequency of fatty-acid binding protein (FABP) within a subject's biological sample (e.g., plasma sample, serum sample, blood sample, urine sample, fecal sample) is used as a biomarker for enterocyte integrity. Such methods are not limited to determining the level, presence, and/or frequency of a particular type of FABP. In some embodiments, the level, presence, and/or frequency of FABP2 are determined. The methods are not limited to a particular manner of determining the level, presence, and/or frequency of FABP (e.g., FABP2) within a subject's biological sample (see, e.g., Examples I and II).

Such methods are not limited to specific biomarkers for paracellular tight junction function. In some embodiments, the level, presence, and/or frequency of claudin within a subject's biological sample (e.g., plasma sample, serum sample, blood sample, urine sample, fecal sample) is used as a biomarker for enterocyte integrity. Claudins are a family of proteins that are important components of the intestinal tight junctions, where they establish the paracellular barrier that controls the flow of molecules in the intercellular space between the cells of an epithelium. Such methods are not limited to determining the level, presence, and/or frequency of a particular type of claudin (e.g., claudins 1, 2, 3, 4, 5, 6, 7, 8, 9, 10, 11, 12, 13, 14, 15, 16, 17, 18, 19, 20, 21, 22, 23 and/or 24). In some embodiments, the level, presence, and/or frequency of claudin-3 is determined as a biomarker for paracellular tight junction function. The methods are not limited to a particular manner of determining the level, presence, and/or frequency of claudin (e.g., claudin-3) within a subject's biological sample (see, e.g., Examples I and II).

Such methods are not limited to specific biomarkers for gut inflammation. In some embodiments, the level, presence, and/or frequency of calprotectin within a subject's biological sample (e.g., plasma sample, serum sample, blood sample, urine sample, fecal sample) is used as a biomarker for gut inflammation. Calprotectin is an abundant neutrophil protein found in both plasma and stool that is markedly elevated in infectious and inflammatory conditions, including inflammatory bowel disease (see, e.g., Konikoff, et al. Inflamm. Bowel Dis. 2006 June; 12(6):524-34). The methods are not limited to a particular manner of determining the level, presence, and/or frequency of calprotectin within a subject's biological sample (see, e.g., Konikoff, et al. Inflamm. Bowel Dis. 2006 June; 12(6):524-34).

Such methods are not limited to a particular manner of characterizing a subject's intestinal barrier function following determining the level, presence, and/or frequency of biomarkers for intestinal barrier function. In some embodiments, the determined level, presence, and/or frequency of biomarkers for intestinal barrier function (e.g., biomarkers for gut inflammation, biomarkers for functional enterocyte mass, biomarkers for enterocyte integrity, biomarkers for paracellular tight junction function) are compared with established norms for each determined biomarker for intestinal barrier function. Such methods are not limited to particular established norms for intestinal barrier function. Examples include, but are not limited to, an established norm for healthy (non-compromised) intestinal barrier function, non non-healthy intestinal barrier function (compromised), and an established norm for inbetween healthy and non-healthy intestinal barrier function. Such established norms are based upon the level, presence, and/or frequency of the specific biomarkers determined within the specific sub-population. Other established norms include, for example, established norms for particular medical procedures (e.g., a subject undergoing surgical repair or palliation of congenital heart disease (CHD); a subject undergoing cardiopulmonary bypass surgery; a subject with acute respiratory failure, a critical care patient (e.g., human, veterinary animal, etc.) that is being closely monitored), an established norm for exposure to vasopressin and/or epinephrine during a medical procedure, an established norm for undergoing cardiopulmonary surgery.

Such methods are not limited to a frequency of determining the level, presence, and/or frequency of such biomarkers for purposes of characterizing a subject's intestinal barrier function. In some embodiments, such determining could be once a day, every hour, every four hours, every twelve hours, every other day, monthly, annually, continually, etc. In other embodiments, such determining could be as often as by the minute and/or hours. In some embodiments, such determinations occur for as long as a challenge to a subject's intestinal barrier function is being experiences (e.g., a human critical care patient that is being closely monitored; a subject undergoing surgical repair or palliation of congenital heart disease (CHD); a subject undergoing cardiopulmonary bypass surgery). For example, in some embodiments, a subject undergoing cardiopulmonary bypass surgery has its intestinal barrier function characterized as frequently as necessary to ensure optimal intestinal barrier function. Indeed, a higher frequency of intestinal barrier function characterization permits comparison between time points which allows a gauging as to improvement or non-improvement of intestinal barrier function over a period of time.

In certain embodiments, the present invention provides methods for obtaining a subject's risk profile for developing intestinal barrier dysfunction. In some embodiments, such methods involve obtaining a biological sample from a subject (e.g., plasma sample, serum sample, blood sample, urine sample, fecal sample) (e.g., a human subject at risk for developing intestinal barrier dysfunction), determining the level, presence, and/or frequency of biomarkers for intestinal barrier function within the sample (e.g., biomarkers for gut inflammation, biomarkers for functional enterocyte mass, biomarkers for enterocyte integrity, biomarkers for paracellular tight junction function), and generating a risk profile for developing intestinal barrier dysfunction based upon the determined biomarkers. For example, in some embodiments, a generated risk profile will change depending upon the determined biomarkers for intestinal barrier function. The present invention is not limited to a particular manner of generating the risk profile. In some embodiments, a processor (e.g., computer) is used to generate such a risk profile. In some embodiments, the processor uses an algorithm (e.g., software) specific for interpreting the level, presence, and/or frequency of biomarkers of intestinal barrier function as determined with the methods of the present invention. In some embodiments, the biomarkers determined with the methods of the present invention are inputted into such an algorithm, and the risk profile is reported based upon a comparison of such input with established norms (e.g., established norm for intestinal barrier dysfunction, established norm for healthy intestinal barrier function, established norm for various risk levels for developing intestinal barrier dysfunction, established norm for subjects undergoing medical procedures known to compromise intestinal barrier function (e.g., cardiopulmonary bypass surgery)). In some embodiments, the risk profile indicates a subject's risk for developing intestinal barrier dysfunction or a subject's risk for re-developing intestinal barrier dysfunction. In some embodiments, the risk profile indicates a subject to be, for example, at a very low, a low, a moderate, a high, and a very high chance of developing or re-developing intestinal barrier dysfunction. In some embodiments, the risk profile indicates risk based on a population average at a desired level of specificity (e.g., 90%).

Previous research involving children after cardiac surgery demonstrated that biomarkers of intestinal barrier function were found to be improved with delivery of small dose enteral nutrition (EN), and to correlate with symptoms of feeding intolerance. Infants diagnosed with necrotizing enterocolitis, who have a rise in these same biomarker levels after re-introduction of EN were found to have worse outcomes. In these series of patients, biomarkers of intestinal barrier function improve with early EN, correlate with symptoms of feeding intolerance, and are predictive of gastrointestinal complications. In such experiments, intestinal barrier function was also modulated by dose and type of vasoactive infusion, and by mean airway pressure. These data indicate that non-invasive assessment of intestinal barrier function guides safe and optimized EN delivery in children with hemodynamic instability and respiratory failure.

Moreover, the absence of EN results in impaired intestinal barrier function in humans and animals, an effect worsened by Parenteral Nutrition (PN) administration. Intestinal barrier dysfunction is linked to sepsis, hospital-acquired infection risk, and to immunoregulatory responses triggering organ dysfunction. Mice with ischemia reperfusion injury, given PN with 15% of goal EN, retained intestinal barrier function. Determining minimum dose of EN to preserve the intestinal barrier could reduce hospital-acquired infection risk and morbidity after pediatric critical illness for patients who fail to tolerate full EN.

As such, in certain embodiments, a health care provider (e.g., a physician) will use the methods for characterizing a subject's intestinal barrier function in determining a course of treatment or intervention involving EN. For example, in some embodiments, the amount of enteral nutrition (EN) to provide to a subject is based upon the determined intestinal barrier function. In some embodiments, an increased amount of EN is administered to a subject upon detection of compromised intestinal barrier function. In some embodiments, so as to prevent compromised intestinal barrier function, EN is administered prophylactly. In some embodiments, an amount of EN administered to a subject is reduced upon detection of non-compromised intestinal barrier function.

While EN alone is clinically recognized as a superior strategy to parenteral nutrition (PN) alone, EN sometimes fails to provide adequate nutritional support during pediatric critical illness. Experiments described herein investigate the combination of early EN combined with early PN to improve nutritional outcome and intestinal epithelial barrier function for critically ill infants and children (e.g., with acute respiratory failure). While the present invention is not limited to a particular mechanism, it is contemplated that by providing combined EN and PN, the risks of early PN on PICU morbidity is mitigated and the negative effects of acute malnutrition on long-term neuro-cognitive outcome are prevented. Thus, in some embodiments, in order to treat or prevent reduced intestinal barrier function, an early combined EN and PN is administerd.

In some embodiments, at initiation (e.g., upon admission to a critical care unit or initiation of hospitalization), initial nutrition is approximately 75% (e.g., 50%, 55%, 60%, 65%, 70%, 75%, 80%, 85%, 90%, etc) of total calories and enteral nutrition is approximately 25% (e.g., 10%, 15%, 20%, 25%, 30%, 35%, 40%, 45%, 50%, etc.). In some embodiments, the concentrations of EN and PN are reverse titrated such that after a period of approximately 7 days (e.g., 1-14 days), the subject is on 100% EN.

In certain embodiments, the present invention provides kits for characterizing intestinal barrier function in a subject (e.g., a human subject). In some embodiments, such kits include reagents useful, sufficient, or necessary for determining the level, presence, and/or frequency of one or more biomarkers for intestinal barrier function (e.g., biomarkers for gut inflammation, biomarkers for functional enterocyte mass, biomarkers for enterocyte integrity, biomarkers for paracellular tight junction function). In some embodiments, the kits further contain algorithms useful for comparing the determined biomarkers with established norms, and/or generating such established norms.

In certain embodiments, the present invention provides assays for determining the level, presence, and/or frequency of biomarkers for intestinal barrier function (e.g., biomarkers for gut inflammation, biomarkers for functional enterocyte mass, biomarkers for enterocyte integrity, biomarkers for paracellular tight junction function). For example, in certain embodiments, the present invention provides an assay that facilitates the determining of the level, presence, and/or frequency of one or more biomarkers for intestinal barrier function. In some embodiments, the assay is portable. For example, in some embodiments, the assay is a portable “dip-stick” wherein upon exposure to a biological sample from a subject (e.g., exposing the “dip-stick” to a urine sample), a visually detectable event occurs on the “dip-stick” indicating intestinal barrier function. For example, in some embodiments, the “dip-stick” is configured such that a color based event will occur at separate locations on the “dip-stick” indicating intestinal barrier function for specific intestinal barrier function biomarkers. In some embodiments, one event will occur indicating intestinal barrier function status which incorporates multiple intestinal barrier function biomarkers. In some embodiments, the “dip-stick” is configured to indicate compromised intestinal barrier function. In some embodiments, the “dip-stick” is configured to indicate non-compromised intestinal barrier function. In some embodiments, the “dip-stick” is configured to indicate a need for additional enteral nutrition to be administered to a subject. In some embodiments, the “dip-stick” is configured to indicate the amount of additional enteral nutrition to administer to a subject. In some embodiments, the “dip-stick” is configured to indicate a need for a higher amount of additional enteral nutrition to administer to a subject. In some embodiments, the “dip-stick” is configured to indicate a need for maintenance of the amount of enteral nutrition being administered to a subject. In some embodiments, the “dip-stick” is configured to indicate a need for a reduced amount of additional enteral nutrition to administer to a subject.

Any suitable dip-stick device can be utilized according to the present invention. Such dip-stick testing devices are commonly known in the art for testing urine samples for the presence or absence of an analyte. In one particular embodiment, the dip-stick device can be a lateral flow assay device that performs a heterogeneous assay. A heterogeneous assay is one in which a species is separated from another species prior to detection. Separation may be carried out by physical separation, e.g., by transferring one of the species to another reaction vessel, filtration, centrifugation, chromatography, solid phase capture, magnetic separation, and so forth. The separation may also be nonphysical in that no transfer of one or both of the species is conducted, but the species are separated from one another in situ. In some embodiments, for example, a heterogeneous immunoassay is performed that utilizes mechanisms of the immune systems, wherein antibodies are produced in response to the presence of antigens that are pathogenic or foreign to the organisms. These antibodies and antigens, i.e., immunoreactants, are capable of binding with one another, thereby causing a highly specific reaction mechanism that may be used to determine the presence or concentration of that particular antigen in a fluid test sample. In other embodiments, however, the heterogeneous assay may employ non-specific chemical reactions to achieve the desired separation.

As such, the methods of the present invention function as a “decision rule” for administering enteral nutrition to a subject exposed to an intestinal barrier function challenge.

In certain embodiments, the present invention provides methods and materials for identifying mammals (e.g., humans) experiencing intestinal barrier dysfunction by determining the level, presence, and/or frequency of one or more (e.g., preferably two or more) biomarkers for intestinal barrier function (e.g., biomarkers for gut inflammation, biomarkers for functional enterocyte mass, biomarkers for enterocyte integrity, biomarkers for paracellular tight junction function) within a biological sample obtained from the mammal and comparing such determined biomarkers with established norms. Depending on the particular set of markers employed in a given analysis, the statistical analysis will vary. For example, where a particular combination of markers is highly specific for intestinal barrier dysfunction, the statistical significance of a positive result will be high. It may be, however, that such specificity is achieved at the cost of sensitivity (e.g., a negative result may occur even in the presence of intestinal barrier dysfunction). By the same token, a different combination may be very sensitive (e.g., few false negatives, but has a lower specificity). Particular combinations of markers may be used that show optimal function with different ethnic groups or sex, different geographic distributions, different stages of intestinal barrier dysfunction, different degrees of specificity or different degrees of sensitivity. Particular combinations may also be developed which are particularly sensitive to the effect of therapeutic regimens on intestinal barrier progression (e.g., supplemental administration of enteral nutrition). Subjects may be monitored after a therapy and/or course of action to determine the effectiveness of that specific therapy and/or course of action.

In some cases, a matrix marker panel (e.g., two or more selected from biomarkers for gut inflammation, biomarkers for functional enterocyte mass, biomarkers for enterocyte integrity, biomarkers for paracellular tight junction function) can be used to identify mammals experiencing intestinal barrier dysfunction.

In some cases, data can be analyzed using quantified biomarkers of intestinal barrier function (e.g., biomarkers for gut inflammation, biomarkers for functional enterocyte mass, biomarkers for enterocyte integrity, biomarkers for paracellular tight junction function) to create a logistic model, which can have both high sensitivity and high specificity. For example, a logistic model can also incorporate population variables like gender and age to adjust cut-off levels for test positivity and thereby optimize assay performance in a screening setting.

The present invention also provides methods and materials to assist medical or research professionals in determining whether or not a mammal is experiencing intestinal barrier dysfunction and whether supplemental administration of enteral nutrition is required. Medical professionals can be, for example, doctors, nurses, medical laboratory technologists, and pharmacists. Research professionals can be, for example, principle investigators, research technicians, postdoctoral trainees, and graduate students.

After the determining the level, presence, and/or frequency of particular biomarkers in a biological sample is reported, a medical professional can take one or more actions that can affect patient care. For example, a medical professional can record the results in a patient's medical record. In some cases, a medical professional can record a diagnosis of a intestinal barrier dysfunction, or otherwise transform the patient's medical record, to reflect the patient's medical condition. In some cases, a medical professional can review and evaluate a patient's entire medical record, and assess multiple treatment strategies, for clinical intervention of a patient's condition (e.g., supplemental administration of enteral nutrition). In some cases, a medical professional can record a prediction of intestinal barrier dysfunction with the reported indicators. In some cases, a medical professional can review and evaluate a patient's entire medical record and assess multiple treatment strategies, for clinical intervention of a patient's condition (e.g., maintenance of, reduction of, or increase of enteral nutrition administration).

A medical professional can initiate or modify treatment of an intestinal barrier dysfunction after receiving information regarding the level (score, frequency) associated with biomarkers in a patient's biological sample. In some cases, a medical professional can compare previous reports and the recently communicated level (score, frequency) of such biomarkers, and recommend a change in therapy (e.g., maintenance of, reduction of, or increase of enteral nutrition administration).

A medical professional can communicate the assay results to a patient or a patient's family. In some cases, a medical professional can provide a patient and/or a patient's family with information regarding intestinal barrier dysfunction, including treatment options, prognosis, and referrals to specialists. In some cases, a medical professional can provide a copy of a patient's medical records to communicate assay results to a specialist. A research professional can apply information regarding a subject's assay results to advance intestinal barrier function research. For example, a researcher can compile data on the assay results, with information regarding the efficacy of a drug for treatment of intestinal barrier dysfunction to identify an effective treatment. In some cases, a research professional can obtain assay results to evaluate a subject's enrollment, or continued participation in a research study or clinical trial. In some cases, a research professional can classify the severity of a subject's condition, based on assay results. In some cases, a research professional can communicate a subject's assay results to a medical professional. In some cases, a research professional can refer a subject to a medical professional for clinical assessment of intestinal barrier dysfunction, and treatment thereof. Any appropriate method can be used to communicate information to another person (e.g., a professional). For example, information can be given directly or indirectly to a professional. For example, a laboratory technician can input the assay results into a computer-based record. In some cases, information is communicated by making a physical alteration to medical or research records. For example, a medical professional can make a permanent notation or flag a medical record for communicating a diagnosis to other medical professionals reviewing the record. In addition, any type of communication can be used to communicate the information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

In some embodiments, the methods disclosed herein are useful in monitoring the treatment of intestinal barrier dysfunction. For example, in some embodiments, the methods may be performed immediately before, during and/or after a treatment to monitor treatment success. In some embodiments, the methods are performed at intervals on disease free patients to insure treatment success.

The present invention also provides a variety of computer-related embodiments. Specifically, in some embodiments the invention provides computer programming for analyzing and comparing a pattern of biomarkers for intestinal barrier function detection results in a biological sample obtained from a subject to, for example, a library of such marker patterns known to be indicative of the presence or absence of intestinal barrier dysfunction, or a particular stage intestinal barrier dysfunction.

In some embodiments, the present invention provides computer programming for analyzing and comparing a first and a second pattern of detection results of biomarkers for intestinal barrier function from a biological sample taken at at least two different time points. In some embodiments, the first pattern may be indicative of a pre-intestinal barrier dysfunction condition and/or low risk condition for intestinal barrier dysfunction and/or progression from a pre-intestinal barrier dysfunction condition to an intestinal barrier dysfunction. In such embodiments, the comparing provides for monitoring of the progression of the condition from the first time point to the second time point.

In yet another embodiment, the invention provides computer programming for analyzing and comparing a pattern of biomarkers for intestinal barrier function results from a biological sample to a library of intestinal barrier function-specific marker patterns known to be indicative of the presence or absence of intestinal barrier dysfunction, wherein the comparing provides, for example, a differential diagnosis between a pre-intestinal barrier dysfunction status, and full blown intestinal barrier dysfunction (e.g., the marker pattern provides for staging and/or grading of the intestinal barrier dysfunction condition).

The methods and systems described herein can be implemented in numerous ways. In one embodiment, the methods involve use of a communications infrastructure, for example the internet. Several embodiments of the invention are discussed below. It is also to be understood that the present invention may be implemented in various forms of hardware, software, firmware, processors, distributed servers (e.g., as used in cloud computing) or a combination thereof. The methods and systems described herein can be implemented as a combination of hardware and software. The software can be implemented as an application program tangibly embodied on a program storage device, or different portions of the software implemented in the user's computing environment (e.g., as an applet) and on the reviewer's computing environment, where the reviewer may be located at a remote site (e.g., at a service provider's facility).

A system for use in the methods described herein generally includes at least one computer processor (e.g., where the method is carried out in its entirety at a single site) or at least two networked computer processors (e.g., where detected biomarker data for a biological sample obtained from a subject is to be input by a user (e.g., a technician or someone performing the assays)) and transmitted to a remote site to a second computer processor for analysis detection results is compared to a library of patterns known to be indicative of the presence or absence of a pre-intestinal barrier dysfunction condition), where the first and second computer processors are connected by a network, e.g., via an intranet or internet). The system can also include a user component(s) for input; and a reviewer component(s) for review of data, and generation of reports, including detection of a pre-intestinal barrier dysfunction condition, staging and/or grading of intestinal barrier dysfunction, or monitoring the progression of a pre-intestinal barrier dysfunction condition or full intestinal barrier dysfunction. Additional components of the system can include a server component(s); and a database(s) for storing data (e.g., as in a database of report elements, e.g., a library of marker patterns known to be indicative of the presence or absence of a pre-intestinal barrier dysfunction condition and/or known to be indicative of a grade and/or a stage of a intestinal barrier dysfunction, or a relational database (RDB) which can include data input by the user and data output. The computer processors can be processors that are typically found in personal desktop computers (e.g., IBM, Dell, Macintosh), portable computers, mainframes, minicomputers, or other computing devices.

Further embodiments provide compositions and methods for assaying and treating intestinal barrier function.

### EXAMPLES

The invention now being generally described, will be more readily understood by reference to the following example, which is included merely for purposes of illustration of certain aspects and embodiments of the present invention, and are not intended to limit the invention.

### Example I

This example describes the materials and methods for Example II.

### Patients

Parents were approached for signed written consent for all eligible children scheduled for CHD surgery in the pre-anesthesia clinic at Diamond Children's Medical Center in Tucson, Ariz. Inclusion criteria were age greater than 37 weeks corrected gestational age and a diagnosis of CHD requiring surgical repair or palliation with the use of CPB. Exclusion criteria were known pre-existing gastrointestinal dysfunction, immune dysfunction, active intracranial bleeding, and anuric renal failure. All operations were performed by two surgeons. CPB and anesthetic regimens were per usual care.

### Blood Samples

Blood samples for measurement of plasma FABP2, claudin 3, and citrulline were collected from indwelling intravascular catheters pre-operatively after induction of general anesthesia, but prior to CPB, and at 6, 12, 24, 48, and ≧120 hours post-operatively. Final samples at ≧120 hours were collected between 120 and 168 hours post-operatively to evaluate the return to baseline values. Control samples were obtained intra-operatively from the CPB circuits prior to connection to the patient. Blood was collected from arterial catheters, in place for clinical monitoring, immediately placed into K+EDTA (BD Vacutainer, Franklin Lakes, N.J.) collection tubes. Blood and urine samples (below) were immediately stored at 4° C., spun at 3400 rpm for 15 minutes within 4 hours of collection, and plasma stored at −80° C. until analysis.

Clinical data included candidate factors likely to alter intestinal barrier function. Candidate patient and treatment factors were chosen based on literature review. Multiple parameters were collected including; patient demographics and vital statistics, cardiac diagnoses, type of surgical repair, CPB characteristics, anesthetic regimen, hemodynamic variables, laboratory values related to organ function and adequacy of circulation, in addition to fluid and nutritional management characteristics. Vasoactive-inotrope score (VIS) was determined at time of sample collection and was previously validated (see, e.g., Gaies, et al., Pediatric critical care medicine: a journal of the Society of Critical Care Medicine and the World Federation of Pediatric Intensive and Critical Care Societies 2010; 11:234-8).

### Analysis of Plasma FABP2 and Claudin 3 Levels

Plasma concentrations of human FABP2 were evaluated by ELISA according to the manufacturer's protocol (Standards range: 10 000 pg/ml to 156.25 pg/ml) (R&D Systems, Minneapolis, Minn.). Claudin 3 concentration was evaluated by ELISA in plasma from patients according to manufacturer's instruction (standard range from 20 ng/ml to 0.312 ng/ml) (Uscn life science Inc, Wuhan, China).

### Analysis of Plasma Citrulline Levels

### Materials

Citrulline 99+% (Sigma-Aldrich, St. Louis, Mo.) and the internal standard L-Citrulline (5-13C, 99%; 4,4,5,5-D4, 95%-Cambridge Isotope Laboratories, Inc., Andover, Mass.) were obtained.

### Protein Precipitation

50 ng of L-Citrulline (5 μl of 10 μg/μl) were added to 45 μl aliquot of the sample to make a final concentration of 1 ng/μl. Proteins were removed via acetone precipitation: the mixture was vortexed and incubated at −20° C. overnight, then centrifuged at 4° C. for 10 min at 13,000 rpm. Supernatant was removed and dried down to approximately 50 μl. 2 μl were used per injection.

### Liquid Chromatography/Mass Spectrometry

Samples were separated on Phenomenex Luna HILIC 200A SB-C18, 3 μm, 150×2.00 mm column (Torrance, Calif.) using Paradigm MS4B-multi-dimensional separations module (Michrom BioResources, Inc., Auburn, Calif.). The mobile phases 0.1% FA/H₂O (A) and 0.1% FA/ACN (B) were delivered at a flow rate of 200 μl/min with the following gradient: 10% A (0-2.5 min), from 10 to 50% A (2.5-4 min), 50% A (4-8 min), from 50 to 10% A (8-10 min), 10% A (10-16 min). Full injection loop (20 μl) was used to introduce 2 μl sample diluted with 18 μl of solvent B. AB SCIEX API 3000 triple-quadrupole mass spectrometer (Applied Biosystems, Foster City, Calif.) controlled by Analyst 1.5.1 in-line with the HPLC was used for citrulline detection and quantitative analysis. Mass spectrometric analysis was performed using Multiple Reaction Monitoring (MRM-MS) scan type in positive mode with a TurboIonSpray source. The following transitions were used for detection and quantitation: for citrulline m/z 176.1→159.2 for quantitation and 176.1→69.9 for confirmation (collision energy CE 14 and 35V respectively; collision cell exit potential CXP, 12 and 11V). For the internal standard quantitation—m/z 181.2→164.4 and 181.2→75.0 for confirmation (collision energy CE 14 and 35V respectively; collision cell exit potential CXP, 12 and 11V).

### Analysis of Plasma Cytokines

Baseline and serial Th-1 and Th-2 cytokine responses known to impact intestinal epithelial tight junction function and enterocyte health in animal models of CPB were analyzed. Level of IL-8, TNF-α, IL-6, IL-4, IL-1α, IL-1β, IL-17α, IL-12p70 and p40, and IL-10, IFN-γ were evaluated using an xMAP assay (Millipore, Billerica, Mass.) and Luminex 100 platform (Liquichip; Qiagen, Valencia, Calif.)

### Dual Sugar Permeability Testing

Subjects were given 2 ml/kg of lactulose/mannitol (lactulose 5 μm/100 ml+mannitol 2 μm/100 ml) solution via nasogastric tube (NGT) in the operating room after induction of general anesthesia. This test was repeated at 48 and ≧120 hours either orally of via NGT, if in place. Urine was collected for 6 hours, placed in a urine collection tube with preservative (boric acid), and further processed similar to blood samples. Not all patients completed the DSPT at 48 and 120 hours due to refusal to drink sugar solution, or if they were discharged to home before the study day. Lactulose and mannitol in urine were determined by HPLC at Baylor College of Medicine, as previously described (see, e.g., Shulman, et al., PediatrRes 1998; 44:519-23).

### Data Analysis

Data were analyzed using STATA SE/11 software. Raw numbers were reported for type of cardiac diagnosis and surgical procedure performed. For continuous variables with normal or skewed distributions we report means with standard errors, or medians with interquartile ranges, respectively. As citrulline values were normally distributed, the effect of binary variables on citrulline were evaluated with a Student's t-test. When comparing the relationship of two continuous variables we report the results of pairwise correlations, with missing values handled by pairwise deletion. The effect continuous and categorical variables over time on repeated log-transformed plasma FABP2, log-transformed claudin 3, and citrulline was evaluated with multi-level mixed-effects linear regression. The effect of categorical variables on peak plasma FABP2, peak claudin 3, and lowest citrulline values were evaluated with Kruskal Wallis. The effect of binary variables on peak plasma values for FABP2 and claudin 3 was tested with Mann-Whitney ranksum, and with the Student's t-test for citrulline. We compared subjects with high (>50^{th} percentile) FABP2, claudin 3, and citrulline threshold values to identify characteristics or clinical factors associated with high plasma values of each biomarker with Mann Whitney ranksum. A p value below 0.05 was considered significant for all tests.

### Example II

Baseline and serial post-operative plasma FABP2, citrulline, claudin 3, Th-1 and Th-2 cytokines, and DSPT were measured in 20 children undergoing repair or palliation of CHD with the use of CPB. Table 1 lists the cardiac diagnoses and operations performed. Patient demographics, cardiac bypass duration, and hospital length of stay are listed in Table 2. Creatinine clearance remained normal for all study subjects. Patients were prescribed intermittent intravenous or oral furosemide post-operatively and maintained >1 ml/kg/hour of urine output. Mean cumulative fluid balance at post-operative day 5 was −300 ml (SEM 255 ml). None of our study patients received pre-operative steroids. Perioperative antibiotic [second generation Cephalosporin (Cefuroxime)] was administered in all patients except in cases of documented penicillin allergy, or history of methicillin resistant S. aureus infection. During sample collection, blood collection occurred during steady state time periods for vasoactive infusions. At the time of sample collection, all subjects were within one standard deviation of age-appropriate mean arterial pressures. Several patients had caudal or spinal morphine as part of their post-operative pain management. Caudal morphine or bupivicaine were not associated with changes in post-operative FABP2, claudin3, or citrulline (p>0.05).

TABLE 1
Patient Demographic Information

| Patient characteristic | N = 20 | |
|---|---|---|
| Median age, months (IQR) | 17 | (3.1-76.9) |
| Median weight, kg (IQR) | 9.3 | (6.6-22.5) |
| Male gender, N (%) | 14 | (70) |
| Median CPB time, min (IQR) | 102 | (78-126) |
| Mortality, N (%) | 0 | (0) |
| Median hospital length of stay, days (IQR) | 15 | (7-31) |

IQR, interquartile range;
kg, kilogram;
CPB, cardiopulmonary bypass.

TABLE 2
Surgical Diagnoses and Operation Performed

| Diagnoses | N | Operation | N |
|---|---|---|---|
| TOF | 2 | TOF repair | 2 |
| Septal defects | 8 | Tricuspid valve repair | 1 |
| AV canal defects | 3 | Arterial switch | 1 |
| Aortic/subaortic stenosis | 4 | Fontan revision | 1 |
| Coarctation of the | 1 | RVOT repair | 2 |
| aorta/hypoplastic arch | | | |
| Mitral valve defects | 2 | Septal defect repair | 8 |
| TAPVR | 1 | RV-PA conduit | 5 |
| Pulmonary stenosis/atresia | 3 | Subaortic/aortic stenosis repair | 3 |
| Pulmonary insufficiency | 3 | Coarctation/hypoplastic arch repair | 1 |
| DORV | 2 | Mitral valve repair | 2 |
| Ebstein's anomaly | 1 | Ross procedure | 1 |
| | | AV canal repair | 1 |
| | | TAPVR repair | 1 |

AV, atrioventricular;
TAPVR, total anomalous pulmonary venous return;
DORV, double outlet right ventricle;
RVOT, right ventricular outflow tract;
RV-PA, right ventricle to pulmonary artery.

Total number of diagnoses and operations performed exceeds 20 as some patients had multiple defects which were repaired.

### Baseline Plasma Biomarker

At baseline, children with congenital heart disease had >10-fold higher mean (SEM) plasma FABP2 levels, 3815 (976) pg/ml compared to healthy children (normal range 41-336 pg/ml) (see, e.g., Derikx, et al., PloS one 2008; 3:e3954). Subjects had normal baseline mean (SEM) plasma citrulline, 28.9 (2.2) μmol/L [normal range (14-39 μmol/L)²⁴], and claudin 3 mean (SEM), was 3.7 (2.0) ng/ml. Differences were not found between type of cardiac lesion and any plasma biomarkers either at baseline or post-operatively. There was no association between left ventricular outflow tract obstruction, septal defects, or presence of a persistent ductus arteriosus and plasma FABP2, claudin 3, or citrulline (p>0.05). Significant differences were not found in the plasma FABP2, claudin 3, or citrulline with surgical transition from hypoxemic to normoxemic circulation, or with correction of shunt (p>0.05).

### Serial Plasma FABP2

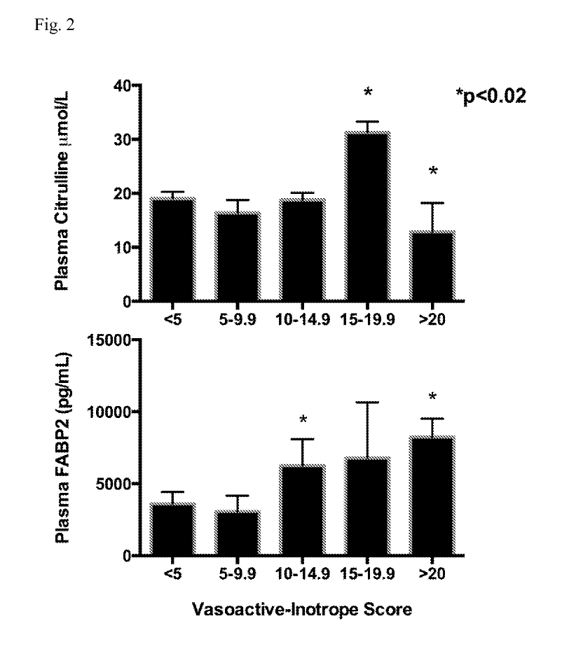

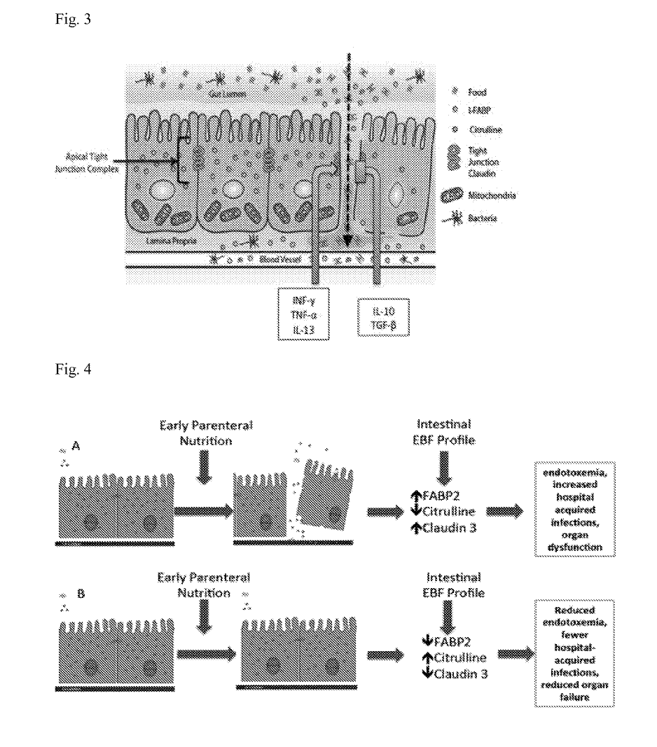

Patient and treatment factors were evaluated over time, which were associated with elevated (>50^{th}%) serial FABP2 levels. High FABP2 was associated with higher mean airway pressure and higher VIS, but not with hypotension, hypertension, SVC-O₂ saturation, or fluid balance. Peak post-operative plasma FABP2 was associated with CPB duration (p=0.01). Elevation in plasma FABP2 over time was associated with use of epinephrine and vasopressin infusions, but not with dopamine or milrinone at any dose (p<0.01) (FIG. 2) Epinephrine was associated with elevated plasma FABP2 (p=0.03). In the study population, vasopressin was used only after initial epinephrine titration for subjects with low cardiac output syndrome. Subjects with VIS>20, who all received combined vasopressin and epinephrine had the highest plasma FABP2 levels (p=<0.01) (FIG. 2). Any enteral nutrition (EN) by post-operative day 2 was associated with improved (decreased) plasma FABP2 levels (p=0.02).

### Serial Plasma Citrulline

Citrulline levels >18 μmol/L (median) were associated with higher SVC-O₂ over time (p=0.01), but not with mean arterial pressure, oxygen saturation, or fluid balance (p>0.05). In univariate analysis. VIS score was not found to be associated with citrulline levels. However, in mixed effects linear regression with time nested within patient ID, citrulline levels were associated with VIS with low citrulline levels associated with VIS>20 (p<0.02), indicating lower functional enterocyte mass at high VIS (FIG. 2).

### Serial Plasma Claudin 3

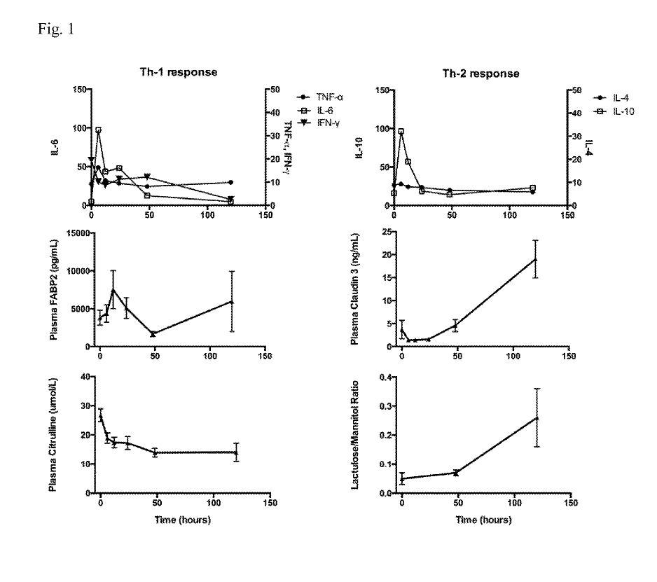

Plasma claudin 3 levels did not rise after CPB, but were elevated remote from CPB at ≧120 hours (FIG. 1). Claudin 3 levels were associated with positive patient fluid balance(p=0.03), symptoms of feeding intolerance (emesis, nausea, abdominal distention, gastrointestinal hemorrhage) (p=0.01) and duration of antibiotic treatment (p=0.02), but not with CPB time, mean airway pressure, hypotension, hypertension, or VIS (p>0.05). Consistent with tight junctions as the primary determinants of intestinal epithelial barrier permeability, claudin 3 values correlated with repeated lactulose/mannitol ratios over time, (p<0.01) (FIG. 1).

### Dual Sugar Permeability Testing

On a subset of patients dual sugar permeability testing (n=12) was performed, and evaluated small intestinal permeability at baseline, then at 48 and ≧120 hours (FIG. 1). Lactulose/mannitol ratios were associated with claudin 3 levels over time and rose remote from CPB (p<0.01). Mannitol was a routine component of CPB prime in our study subjects, but as our DSPT testing occurred either prior to CPB or at 48 and 72 hours, mannitol given with CPB should not impact our results.

### Th-1 and Th-2 Cytokine Profiles

Differences over time were not found after CPB for IL-1β, IL-17α, IL-12p70 and p40. Results for Th-1 (IFN-γ, IL-6, TNF-α, IL-1α) and Th-2 (IL-4, IL-10) cytokines (FIG. 1) are reported. In univariate analysis, higher FABP2 levels were associated with IL-10, IL-6, IL-8, and TNFα. In linear mixed effects models examining the interaction between repeated cytokine and FABP2 levels over time, significant associations between FABP2 and IL-10, IL-6, IL-8, and TNF-α were found. Claudin 3 levels over time were associated with IL-1α, but not with TNF-α. No association between plasma citrulline and Th-1 or Th-2 response was found. Bypass time had a strong positive correlation with peak IL-6 and IL-8 levels, r=0.63 and 0.78, respectively. Bypass time had a moderate negative correlation with IL-4 and IFN-γ levels, r=−0.35 and −0.42, respectively.

### Example III

This Example describes studies into the impact of parenteral nutrition (PN) on outcome of patients.

Discharge functional outcomes after organ dysfunction were monitored for >40,000 consecutive PICU admissions. It was found that pre-existing gastrointestinal dysfunction increased the odds of multiple organ dysfunction syndrome (MODS) and increased length of stay⁴⁷. These long stay patients require innovative strategies to improve morbidity such as optimized, early nutritional support.

Enteral and parenteral calorie and protein delivery for 258 children mechanically ventilated for >72 hours and admitted to the PICU at Diamond Children's Medical Center (DCMC) in 2011-2014 was monitored. Prior to implementation of an early enteral nutrition (EN) protocol, children on EN alone received only 20% of goal calories on hospital day 3 by the enteral route alone. After implementation of an Early EN protocol in 2014, calories delivered via the enteral route improved to 55% of goal energy by hospital day 3. Patients in the early PN study on the early EN protocol achieved 100% goal energy by the enteral route by hospital day 2.

### Example IV

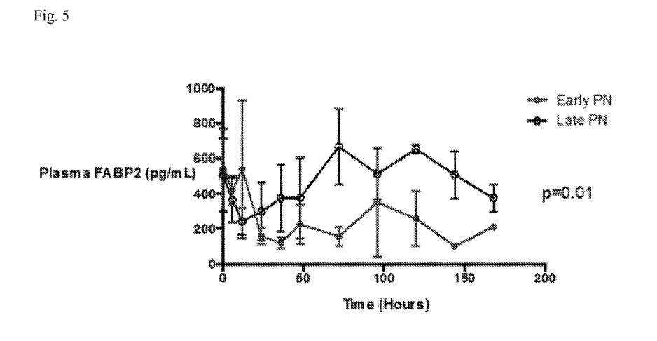

The Example describes a study of supplemental parenteral nutrition for pediatric respiratory failure. In this study, 8 children with acute respiratory failure, who were randomized to early vs. late PN over the initial 7 days of PICU hospitalization, were evaluated. The study protocol compares best practice early EN guidelines to best practice early EN guideline+early PN. In the early PN group, patients achieved nearly immediate early goal calories and protein (begun within 24 hours) via supplementation of advancing EN with Clinimix E and IL (Baxter, Deerfield, Ill.). Patients in the early PN group are often off PN by 48 hours and only resume PN for periods where EN is at less than 80% of goal. The study evaluated the impact of early PN on intestinal epithelial barrier function using minimally invasive plasma biomarkers of intestinal epithelial barrier function. It was found that early PN, independent of calories delivered was significantly associated with improved enterocyte and tight junction integrity, as measured by FABP-2 (p=0.01) and Claudin 3 (p=0.02) plasma concentrations (FIG. 5). Improved plasma citrulline concentrations (p=0.09), consistent with improved intestinal enterocyte functional cell mass, were observed. These findings are consistent with the model shown in FIG. 4B and support the hypothesis that PN, when provided early in the course of acute respiratory failure in children causes a rapid and persistent improvement in intestinal epithelial barrier function. The present disclosure is not limited to a particular mechanism. Nonetheless, it is contemplated that the first 48 hours of critical illness represent a reversible window to rapidly improve intestinal epithelial barrier function. This improved intestinal epithelial barrier function may reduce risk for in-hospital sepsis, new or progressive organ failure, and mortality.

### Example V

This example describes the study of combined early enteranl nutrition (EN) with parenteral nutrition (PN) on intestinal barrier function nutritional outcome for infants and children with acute respiratory failure.

A prospective, single-blind, randomized pilot trial to determine the effect of early PN versus late PN supplementation on proportion of goal energy and calories delivered for infants and children with acute respiratory failure is performed. The primary outcome is improvement of the percentage of goal calories and protein delivered through the first week of PICU hospitalization.

A determination of the effect of early PN on intestinal epithelial barrier function and endotoxemia for infants and children with acute respiratory failure randomized to early vs. late PN supplementation is also conducted. Serial assessment of intestinal epithelial barrier function profile and endotoxemia over the first week of PICU hospitalization are investigated. Intestinal epithelial barrier function profile is measured with minimally invasive plasma tests for functional enterocyte mass (citrulline), enterocyte integrity (FABP-2), paracellular tight junction function (Claudin 3), and intestinal permeability (dual sugar permeability tests). To control for local inflammation, fecal calprotectin is also assayed. Plasma endotoxin concentrations and two-sugar permeability testing are performed to serve as functional assays of intestinal epithelial permeability. Two sugar permeability testing measures intestinal permeability^{68;108}. The co-ingestion of lactulose and mannitol controls for gastric emptying, intestinal fluid volume, intestinal transit time, and overall fluid status, allowing measurement of site-specific paracellular absorption in the gastrointestinal tract¹⁰⁹. These tests rely on paracellular intestinal absorption, destruction in the intestinal tract (site specificity), and renal clearance¹⁰⁹. Normal values are known for infants, children, and adolescents¹⁰⁸. Measurement of plasma endotoxin concentrations with the limulus amebocyte assay (LAL) allows for confirmation of translocation of bacterial products as a result of improved (or worsened) intestinal epithelial barrier function.

Study Details:

Inclusion Criteria
1. Admitted to study hospital pediatric intensive care unit (PICU),
2. Male or Female children one year to 16 years of age,
3. Exhibits Acute Hypoxemic Respiratory Failure as defined as:
   a. Worse PaO2/FiO2≦300 or SpO2/FiO2≦260
   b. No evidence of cardiac dysfunction
   c. Mechanically ventilated,
4. Requires artificial nutrition,
5. Anticipate placement of central venous line within 24 hours of admission.

Exclusion Criteria

Patients will be excluded if at screening they have one or more of the following:
1. Patient transferred from an outside facility and mechanically ventilated >24 hours,
2. Receiving parenteral nutrition for >24 hours
3. Known allergy to lactulose or mannitol,
4. Pregnant,
5. Admit BMI>30,
6. Pre-existing diagnosis for severe gastrointestinal disease (i.e. Crohns, ulcerative colitis, graft versus host disease)
7. Thoracic trauma, abdominal trauma, and/or active intracranial bleeding,
8. Anuric renal failure, previous bowel surgery and/or short gut syndrome,
9. Liver failure or hepatic coma
10. Severe dehydration at time of study randomization
11. Cannot be enterally fed within 24 hours of admission according to the admitting physician,
12. On extracorporeal membrane oxygenation (ECMO),
13. Expected survival <24 hours or limitations to aggressive ICU care (DNR),
14. Receiving active CPR when admitted to the PICU,
15. A pre-existing bronchopleural fistula,
16. Previously enrolled and randomized into this protocol,
17. Actively enrolled in another clinical trial which at the discretion of the PI would conflict with this study.

The study includes seven days of active study intervention. Full study participation completes at 28 days with vital status follow up by phone.

### Randomization of Study Subjects:

Within 24 hours of admission to the PICU or initiation of mechanical ventilation. As age and malnutrition are likely to impact the primary outcome measure, treatment assignments are stratified by age (<3yo, 3-10 yo, 10-<16 yo) and Body Mass Index (BMI) z score (<−2, −2−+2, >2).

### Early EN Protocol:

All study patients (both early and late PN arms) will begin EN, if not already begun prior to randomization, and advanced on EN per the PICU nutrition protocol. Study subjects are placed on an age appropriate peptide based, or hydrolyzed enteral formula within 6-12 hours of admission, with an advancement protocol to achieve goal calories within 48 hours if tolerated, and limit feed interruptions⁷⁵. Feeds may be nasogastric or nasoduodenal and are left to the discretion of the attending physician. Each EN guideline advances EN every 3-4 hours with a goal to reach goal EN rates by 48 hours. For any EN hold of greater than 2 hours the site study team is notified by the bedside nurse to discuss appropriateness of holding EN with the medical team. In this manner avoidable interruptions to EN are avoided. Management of EN intolerance and a bowel regimen are standardized. Bowel regimens are begun the same day as EN. If full volume enteral feeds fail to provide 2-3 gm/kg/day protein, whey protein is added to EN to deliver goal protein. Protein supplementation is begun on day 1 of study protocol.

### Measured Resting Energy Expenditure:

Predictive equations fail to accurately estimate energy requirements in critically ill children, and result in complications related to both under and overfeeding^{69;77;78}. Indirect calorimetry or Schofield equation estimated REE is used to determine energy prescription (1.0-1.2MREE). MREE is delayed until resolution of the following criteria, if present, as they confound MREE measurement: >20% endrotracheal tube air leak, chest tube with air leak, >0.6 fraction of inspired oxygen (FiO2), and metabolic or hemodynamic instability requiring ongoing resuscitation^{69;77;79-81}. All patients who cannot undergo MREE will have estimate REE determined by Schofield equation.

### Early PN (Clinimix and Clinimix E 5/15) Arm:

To ensure similar prescription of energy and protein across study sites, sites have agreed to the following study protocol for macronutrient prescription. Patients randomized to early PN begin PN at 75% of Schofield equation estimated REE and 1.5 gm/kg/day protein within 2 hours after randomization. As determined in our pilot study, most study patients will be managed with Clinimix E 5%/15% to achieve their macronutrient goals and limit fluid overload. No PN regimen will exceed maintenance fluid requirements. Should the patient's care team ask for fluid restriction at less than maintenance fluid rates, calories are restricted to remain within fluid targets. Consistent with national guidelines, patients 100-120% of MREE and 2-3 gm/kg/day protein with combined PN and EN by study day 2, not to exceed 1600 mL/M²/day²¹. Fat calories are delivered in the form of IL to achieve approx. 30% of goal calories by fat. The bedside nurse titrates PN to maintain goal calories and protein every 4 hours with aid of a bedside chart, updated daily and generated by the PICU pharmacist, which lists corresponding PN and EN rates. This titration procedure is routinely used to limit and titrate total hourly intravenous and oral fluid delivery in both PICU's, and is familiar to ICU nursing staff. Delivery of protein and calories remains nearly constant throughout each day although the route of delivery changes by clinical scenario (e.g. NPO for a procedure) and over time as patients advance on EN. Patients resume PN for any 4-hour period less than 60% goal calories during the 7-day study intervention. In this manner patients in the early PN arm receive nearly constant goal energy and protein delivery over the 7 day study intervention.

Late PN Arm (Standard Care with Early EN and Late PN):

Patients randomized to late PN begin PN on study day 5, if failing to meet 60% of MREE on enteral feeds alone. This is consistent with current standard practice for delivery of PN to critically ill infants and children with acute respiratory failure.

### Collection of Biological Samples, Intestinal Permeability Testing, and Timing of the Metabolic Cart:

Collection of blood, urine, and stool provide the basis for intestinal biomarker and microbiome analysis. Eleven blood samples, 4 urine samples, and approximately 7 stool samples are collected over the course of seven days. Research coordinators or bedside personnel collect biological samples.

### Repeated Measures of Intestinal Epithelial Barrier Function:

Assays for I-FABP, Citrulline, and Claudin 3 are performed as described in Examples I and II above. FABP-2/IFABP (R & D Systems) and Claudin 3 (antibodiesonline) are measured by commercially available Enzyme-Linked Immunosorbent Assay (ELISA) kits. Serum citrulline is measured by High Performance Liquid Chromatography (HPLC). Blood is collected from intravascular catheters, in place for clinical monitoring, immediately placed into K+EDTA collection tubes. Urine is collected in 5-9 mL aliquots at the required sample times, placed into urine collection tubes with preservative (boric acid chip. Blood and urine samples are immediately stored at 4° C., spun at 3400 rpm×15 minutes within 4 hours of collection, and stored at −80° C. until analysis. Stool is collected by spontaneous void into diaper or bedpan, placed in preservative free collection bags, and frozen at −80° C. until analysis.

### Measurement of Plasma Endotoxin Concentrations:

Serial Endotoxin Concentrations from blood samples collected as described daily for 5 consecutive study days are measured with the Limulus Amebocyte Assay (LAL) (Hycult Biotech)

### DNA Extraction and Sequencing:

For all days where study patients have a spontaneous fecal sample during study days 1-7, a fecal sample is obtained for fecal calprotectin and microbiome analysis. DNA extraction and sequencing follow the methods described in Caporaso, et. al. ISJME 2012. DNA is extracted using the MoBio Power Soil DNA Isolation Kit (MoBio Laboratories, Inc. Carlsbad, Calif.). Appropriate positive and negative control samples are run to identify contamination, if present. DNA is extracted upon arrival at the site and frozen at −80° C. until sequencing. Extracted DNA is PCR amplified with barcoded primers targeting the V4 region of 16S rRNA with appropriate controls and with standard techniques to avoid contamination of samples. The 16S library is quantified by qPCR. Sample concentration are brought to 2 nM and the MiSeq protocol is followed per manufacturers instructions for preparation of the library for sequencing. Sequences are generated on a MiSeq platform (Illumina, San Diego, Calif.) with a 15-30% PhiX control and pair-ended 2×150 bp protocol. Quality filtering of reads follows the manufacturers specifications for the MiSeq platform.

### Intestinal Permeability Testing:

On study days 1 and 5 (Hour 0 and Hour 96) patients are given 2 mL/kg of a lactulose 5 μm/100 mL and mannitol 1 gm/100 mL oral solution via nasogastric or nasoduodenal tube. Samples are processed as described above, frozen, and shipped to the Texas Children's Hospital department of pathology, where the urine samples are analyzed.

### Efficacy Assessments:

Baseline data includes Body Mass Index (BMI), Pediatric Risk of Mortality (PRISM) score to assess severity of illness82, the Nutritional Index (NI), weight and recumbent length for age, head circumference, and clinical and demographic data. Children are classified as acute or chronic malnutrition by weight for age and height for age BMI z score, respectively.

The primary nutritional outcome is percent of cumulative goal energy and protein delivered during the first week of PICU hospitalization. Delivered EN and PN is recorded into the study log every 4 hours and reasons for interruptions to either EN or PN are identified and recorded. The secondary nutritional outcomes are the mid upper arm circumference and the serial measurement of the modified Prognostic Inflammatory and Nutritional Index (NI) on study day 1 and 5, which incorporates the ratio of C-Reactive Protein and fibrinogen to transferrin and prealbumin41;42. The NI is an outcome measure of nutrition in trials of pediatric critical illness5;41. The NI evaluates sequential biochemical indices of nutrition while controlling for changes in the magnitude of the acute phase reaction, and is a quantitative method to monitor the relationship between nutritional markers and acute phase proteins42. Additional secondary outcomes are the percentage of daily goal calories delivered, organ failure-free days, and change in admission to discharge Pediatric Overall Performance Category (POPC) and Pediatric Cerebral Performance Category (PCPC) scores-measures of functional status during pediatric critical illness. Percentage of goal calories and protein by both enteral and parenteral routes is measured daily through study day 7. Organ dysfunction definitions are by International Pediatric Sepsis Consensus Conference (IPSCC) criteria83. Symptoms of feeding intolerance, use of pressors, fluid overload, reasons for discontinuation of withholding of enteral feeds, type of EN, caloric density of EN are monitored. Indices of growth (e.g., weight and height) are monitored although they are unlikely to change with this brief intervention and are confounded by shifts in fluid status. Adverse events are monitored for 14 total study days. Vital status is monitored at 28 days via phone conversation with parent/guardian or if still in the hospital, by an in person visit.

Anthropometrics: Admission weight and length, daily weight, admission mid upper arm circumference, day 7 mid upper arm circumference, PICU discharge mid upper arm circumference. Mid upper arm circumference is reliably measured between providers and is more resistant to changes in fluid status.

Clinical data which may act as effect modifiers of intestinal epithelial barrier function such as: daily data regarding use of antimicrobials, vasoactive-inotrope score, NIRS monitoring if used, paO2, SaO2, SVC-O2 saturation, systolic blood pressure, diastolic blood pressure, mean arterial blood pressure, central venous pressure if measured via CVL in SVC-RA junction, ventilator parameters (mode of ventilation, mean airway pressure, PIP, FiO2), presence of constipation or diarrhea, use of probiotics/prebiotics are collected.

Statistical Methods (for Analyzing Primary & Secondary Endpoints):

Differences between cumulative percent of goal energy and protein are assessed and means and standard deviations are reported as standard descriptive statistics. Results are considered significant at p<0.05. Relationships between cumulative energy and protein delivered with clinical characteristics, mid upper arm circumference changes, and weight for age z scores will be reported by Pearson's correlation coefficient.

Differences between FABP2, Claudin 3, Citrulline, Lactulose/Mannitol ratios, and Fecal calprotectin are assessed using the following descriptive plots: serum biomarker (y-variable) over time (x-variable) are fit, with linear and smoothing splines. Plots are qualitative. Splines allow examination of possible nonlinear relationships and can be used to specify nonlinear or change-point models. One-way repeated measures Anova with the intestinal barrier assays as continuously measured outcomes and time (1, 2, 3, 5, and 7 days) are used as explanatory variables. If the slope of the serum biomarkers is linear, then change in the slope over time is calculated using a linear mixed effects model with an AR(1) variance-covariance structure. The variance-covariance structure allows for measurements taken closer together to be more similar than those farther apart in time, and adjusts the standard errors for this correlation. If the relationship between the serum biomarker is not linear in time, then change-point or nonlinear mixed effects models are used. Multivariable regression analysis evaluate possible effect modification, or mediation, for non-invasive measures of intestinal epithelial barrier function. Effect modifiers are the interactions with PN group; mediators are those variables that could have a potential additive effect with the PN group. Additionally any potential confounding that might be present is assessed by assessing the change in the parameter estimate for the PN group effect. Potential modifiers, mediators, and confounders, include clinical variables, center effects, and severity of illness. Associations between measures of intestinal function and clinical variables, severity of illness, as well as dose and route of nutrition are assayed.

Microbiome Analysis:

Sequences are assigned to 97% ID Operational Taxonomic Units (OTU's) using a OTU picking protocol in the Quantitative Insights Into Microbial Ecology (QIIME) toolkit with uclust (Edgar 2010) to search sequences against the Greengenes database. Reads without 97% ID are discarded. QIIME is an open source statistical software package for analysis of bacterial 16S rRNA gene sequence data. With the QIIME pipeline the alpha diversity (within samples) and beta diversity (between samples) is determined. Chaol metric is determined to describe the microbial species richness within samples. Shannon index is calculated to describe the species alpha diversity. Weighted Unifrac (beta diversity) distances are calculated between all samples in each replicate, and principal coordinates analysis are applied to visualize the results. Principal Components Analysis (PCA) allows complex microbiome data to be visualized across two or three dimensional scatterplots where the distance between points represent how different the samples are from on another. The principal coordinates each represent a portion of the variability observed between samples. PCA is applied to visualize the beta diversity between participating sites and by duration and type of antibiotic use. Multivariate statistics is used to evaluate the clustering patterns from PCA analysis to compare the distribution of within group differences to the distribution of between group differences with regard to type and duration of antimicrobial prophylaxis regimen, with the following possible covariates either known or suspected to influence microbiome diversity: type of formula (breastmilk, whole protein, hydrolyzed protein formula), duration of NPO status, use of parenteral nutrition, age, sex, race/ethnicity, birth method, previous antibiotic exposures, and use of pre/probiotics, and vasoactive infusions. It is determined if fecal calprotectin, as a measure of local colonic inflammation, is a determinant of alpha diversity of the intestinal microbiome.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method for characterizing intestinal barrier function in a subject comprising:
a) providing
reagents necessary for determining the level, presence, and/or frequency of two or more biomarkers for intestinal barrier function within a biological sample, and
an algorithm
configured to receive information regarding the level, presence and/or frequency of two or more biomarkers for intestinal barrier function within a biological sample obtained from a subject, and
configured to compare received information regarding the level, presence and/or frequency of two or more biomarkers for intestinal barrier function with established norms for intestinal barrier function, and based upon such comparison, characterize the intestinal barrier function for the subject;
b) obtaining a biological sample from a subject;
c) determining the level, presence, and/or frequency of two or more biomarkers indicative of intestinal barrier function within the biological sample;
d) inputting the determined level, presence, and/or frequency of the two or more biomarkers into the algorithm; and
e) characterizing the intestinal barrier function of the subject with the algorithm.

2. The method of claim 1, wherein the subject is a human subject.

3. The method of claim 2, wherein the human subject is a critical care patient that is being closely monitored.

4. The method of claim 3, wherein the critical care patient is undergoing surgical repair or palliation of congenital heart disease (CHD) and/or undergoing cardiopulmonary bypass surgery.

5. The method of claim 1, wherein the two or more biomarkers for intestinal barrier function are selected from the group consisting of a biomarker for functional enterocyte mass, a biomarker for enterocyte integrity, a biomarker for paracellular tight junction function, and a biomarker for gut inflammation.

6. The method of claim 5, wherein the biomarker for functional enterocyte mass is citrulline.

7. The method of claim 5, wherein the biomarker for enterocyte integrity is the fatty-acid binding protein (FABP) FABP2.

8. The method of claim 5, wherein the biomarker for paracellular tight junction function is claudin-3.

9. The method of claim 5, wherein the biomarker for gut inflammation is calprotectin.

10. The method of claim 1, wherein the biological sample is selected from the group consisting of a blood sample, a plasma sample, a serum sample, a fecal sample, and a urine sample.

11. The method of claim 1, wherein the established norm for intestinal barrier function is one or more established norm selected from the group consisting of
an established norm for normal intestinal barrier function specific for the received information regarding the level, presence and/or frequency of two or more biomarkers,
an established norm for compromised intestinal barrier function for the received information regarding the level, presence and/or frequency of two or more biomarkers, and
an established norm for neither healthy nor compromised intestinal barrier function for the received information regarding the level, presence and/or frequency of two or more biomarkers.

12. The method of claim 11, wherein the established norm is specific for medical procedure selected from the group consisting of surgical repair or palliation of congenital heart disease (CHD) and cardiopulmonary bypass surgery.

13. The method of claim 1, wherein steps b), c), d) and e) are repeated for purposes of monitoring the intestinal barrier function for a subject.

14-26. (canceled)

27. A method for treating and/or preventing intestinal barrier dysfunction in a subject, comprising
characterizing a subject's intestinal barrier function with the method described in claim 1,
administering enteral nutrition to the subject if the subject's intestinal barrier function is characterized as non-healthy.

28. The method of claim 27, wherein said enteral nutrition is administered with parenteral nutrition.

29. The method of claim 28, wherein said parenteral nutrition in inititated at the same time as enteral nutrition.

30. The method of claim 29, wherein the concentration of parenteral nutrition is 75% of total calories and the concentration of enteral nutrition is 25% of total calories at the beginning of said treatment.

31. The method of claim 30, wherein the concentration of parenteral nutrition is decreased over time and the concentration of enteral nutrition is increased over time such that at the end of approximately 1 week, all of said nutrition is enteral.

32-35. (canceled)

36. A kit for characterizing intestinal barrier function in a subject comprising
reagents necessary for determining the level, presence, and/or frequency of two or more biomarkers for intestinal barrier function, and
an algorithm
configured to receive information regarding the level, presence and/or frequency of two or more biomarkers for intestinal barrier function within a biological sample obtained from a subject, and
configured to compare received information regarding the level, presence and/or frequency of two or more biomarkers for intestinal barrier function with established norms for intestinal barrier function, and based upon such comparison, characterize the intestinal barrier function for the subject.

37. (canceled)

38. The method of claim 1, wherein the determining the level, presence, and/or frequency of two or more biomarkers indicative of intestinal barrier function within the biological sample is accomplished with an assay configured to determine the level, presence, and/or frequency of the two or more biomarkers indicative of intestinal barrier function within the biological sample.

39-57. (canceled)
